(19) United States
(12) Patent Application Publication
(10) Pub. No.: US 2010/0312189 A1
(43) Pub. Date: Dec. 9, 2010

(54) FLEXIBLE CANNULA DEVICES AND METHODS

(75) Inventors: Frederick E. Shelton, IV, Hillsboro, OH (US); Christopher W. Widenhouse, Clarksville, OH (US)

Correspondence Address: Ethicon Endo-Surgery/Nutter, McClennen & Fish LLP, Seaport West, 155 Seaport Blvd., Boston, MA, 02210-2604, US

(73) Assignee: ETHICON ENDO-SURGERY, INC., Cincinnati, OH (US)

(21) Appl. No.: 12/478,862

(22) Filed: Jun. 5, 2009

(51) Int. Cl. A61B 17/34

(52) U.S. Cl. 604/164.03

(57) ABSTRACT

Various devices and methods are provided for dilating tissue. In one embodiment, a trocar cannula is provided and includes an elongate tubular member defining an inner lumen extending therethrough and configured to receive a surgical instrument therethrough. The tubular member is configured to radially expand to increase an inner diameter of the inner lumen and thereby dilate tissue. In one embodiment, the tubular member has an expansion element incorporated therein and the expansion element is configured to maintain the tubular member in a radially expanded position when no instrument is disposed within the inner lumen.

### FIELD OF THE INVENTION

The present invention relates to methods and devices for providing access into a body cavity, and more particularly to a flexible trocar cannula.

### BACKGROUND OF THE INVENTION

Access ports are widely used in medical procedures to gain access to anatomical cavities ranging in size from the abdomen to small blood vessels, such as veins and arteries, epidural, pleural and subarachnoid spaces, heart ventricles, and spinal and synovial cavities. The use of access ports has become more common as they provide minimally invasive techniques for establishing a portal for a number of procedures, such as those involving the abdominal cavity.

A trocar is one type of access post that is commonly used to provide a minimally invasive pathway for accessing a surgical site. Trocars generally include a cutting assembly (or obturator) that is disposed within an outer cannula. The sharp distal end of the cutting assembly, with the cannula disposed therearound, is urged through the skin until it enters the anatomical cavity being penetrated. The cutting assembly is then withdrawn from the cannula, which remains in place to provide a passageway through which access to the anatomical cavity is provided for other surgical devices.

While effective, there can be many disadvantages when using a typical trocar assembly. For example, the size of the access port is related to the size of the cut made through the skin. Therefore, if a large opening is needed for access to a body cavity, a large wound will need to be created. Additionally, the size of the opening into the body is largely dependent on the size of the trocar disposed through the tissue, thus requiring trocars with large diameters to form a large opening through tissue. While devices are available that are adapted to be inserted through tissue and dilate the tissue to increase the size of the opening into the body cavity, these devices lack any type of rigidity necessary to maintain dilation of the tissue, thus requiring additional components, such as a rigid insert, to maintain tissue dilation and prevent the opening through tissue from collapsing.

Accordingly, there is a need for improved methods and devices for providing access into a body cavity.

### SUMMARY OF THE INVENTION

The present invention provides various devices and methods for accessing a body cavity. In one embodiment, a trocar cannula is provided and includes an elongate tubular member defining an inner lumen extending therethrough and configured to receive a surgical instrument therethrough. The tubular member is configured to radially expand to increase an inner diameter of the inner lumen when an instrument is disposed within the inner lumen. The tubular member can have an expansion element incorporated therein and the expansion element can be configured to maintain the tubular member in a radially expanded position when no instrument is disposed within the inner lumen. In one exemplary embodiment, the expansion element can be biased to an expanded position.

The tubular member have a variety of configurations. In one embodiment, the tubular member can include a sheath disposed therearound and configured to retain the expansion element in an unexpanded configuration such that removal of the sheath allows the expansion element to radially expand. The sheath can also include a closed distal end to provide a seal for the tubular member as the tubular member is inserted through tissue. In another embodiment, the tubular member can include an internal sheath disposed therein and configured to provide resiliency to a distal end of the tubular member to prevent inversion of the distal end of the tubular member when the tubular member is in the radially expanded position. The internal sheath can extend from inside the tubular member and around the distal end of the tubular member. In yet another embodiment, a proximal end of the tubular member can be mated to a housing having an inner lumen extending therethrough and aligned with the inner lumen of the tubular member. At least one seal can be disposed in the housing and it can be configured to form a seal around an instrument inserted therethrough and/or to seal the inner lumen when no instrument is inserted therethrough. The tubular member can also be formed in a variety of ways. In one embodiment, the tubular member is formed from a mesh material.

The expansion element can also have a variety of configurations. In one embodiment, the expansion element can be at least one radially expandable ring disposed within a wall of the mesh material of the tubular member. The radially expandable ring can be in the form of, for example, a coiled wire. The radially expandable ring can be configured to expand from a first diameter to a second diameter, for example, that is four times greater than the first diameter. In another embodiment, the expansion element can be in the form of a plurality of pleats formed in the tubular member and extending longitudinally along the tubular member. Each pleat can include a peak having a rib formed thereon and extending longitudinally along the tubular member, and the ribs can be configured to provide longitudinal stiffness to the tubular member. In one embodiment, the pleats can be configured to interlock with one another to maintain the tubular member in an unexpanded position. Insertion of an instrument through the inner lumen of the tubular member can be effective to cause the pleats to unlock to allow radial expansion of the tubular member.

Methods for dilating tissue are also provided, and in one embodiment the method can include inserting a radially expandable cannula into tissue such that an inner lumen of the cannula defines an opening extending through the tissue and into a body cavity, and inserting an instrument through the inner lumen of the cannula. The instrument causes the radially expandable cannula to expand into an expanded configuration wherein a diameter of the inner lumen is increased. The method also includes removing the instrument from the inner lumen of the cannula such that an expansion element integrally formed with the radially expandable cannula maintains the cannula in the radially expanded configuration. In one embodiment, the expansion member can be a radially expandable ring disposed within a wall of the cannula, and the ring can be biased to the expanded configuration. The radially expandable ring can expand from a first diameter to a second greater diameter upon insertion of the instrument through the inner lumen of the cannula. In another embodiment, the expansion member can be in the form of a plurality of pleats formed in the cannula that extend longitudinally along the cannula, and each pleat can include a peak having a rib formed thereon that provides longitudinal stiffness to the tubular member upon insertion of the instrument through the inner lumen of the cannula.

In another embodiment, a method of dilating tissue is provided that includes inserting a radially expandable cannula into tissue such that an inner lumen of the cannula defines an opening extending through the tissue and into a body cavity. The cannula can include at least one expansion element biased to an expanded position. The method further includes removing a sheath disposed around an outer surface of the cannula such that the expansion element expands to cause the radially expandable cannula to expand into an expanded configuration wherein a diameter of the inner lumen is increased.

### BRIEF DESCRIPTION OF THE DRAWINGS

The invention will be more fully understood from the following detailed description taken in conjunction with the accompanying drawings, in which:

FIG. 1 is a perspective view of a housing and a trocar cannula in the form of a radially expandable tubular member extending from a housing and having expansion members incorporated therein that are configured to dilate tissue;

FIG. 2A is a perspective view of one embodiment of an expansion member in the form of an expandable ring shown in an unexpanded configuration;

FIG. 2B is a perspective view of the expandable ring of FIG. 2A in an expanded configuration;

FIG. 3 is a side cross-sectional view of the tubular member of FIG. 1 showing expansion members incorporated therein and a sheath disposed therearound that is configured to retain the expansion members and the tubular member in an unexpanded configuration;

FIG. 4 is a perspective view of the tubular member and sheath of FIG. 3 showing the sheath being split and pulled in a proximal direction such that expansion members in the tubular member are released to radially expand the tubular member;

FIG. 5 is a side cross-sectional view of another embodiment of a tubular member including expansion members incorporated therein and having a sheath disposed therearound that is configured to retain the expansion members and the tubular member in an unexpanded configuration showing the sheath tearing as a device is inserted through the tubular member;

FIG. 6 is a perspective view of the tubular member and sheath of FIG. 5 showing the sheath tearing to release the expandable members to radially expand the tubular member;

FIG. 7 is a side cross-sectional view of another embodiment of a sheath configured to retain expansion members and a tubular member in an unexpanded configuration, the sheath extending along an outer and inner surface of the tubular member;

FIG. 8 is perspective view of the tubular member and sheath of FIG. 7 showing the sheath being pulled in a proximal direction such that the tubular member is partially expanded;

FIG. 9 is a perspective view of the tubular member of FIG. 7 showing the sheath removed from the tubular member and the tubular member in the expanded configuration;

FIG. 10 is a side cross-sectional view of another embodiment of a sheath configured to retain expansion members and a tubular member in an unexpanded configuration, the tubular member including a central tube disposed therethrough to provide additional stability to the tubular member;

FIG. 11 is a side cross-sectional view of another embodiment of a sheath configured to retain expansion members and a tubular member in an unexpanded configuration, the sheath being formed from an outer portion and an inner portion that are joined together at their distal ends

FIG. 12 is a side cross-sectional view of another embodiment of a sheath configured to retain expansion members and a tubular member in an unexpanded configuration, the sheath extending along an outer surface of the tubular member and into the distal end thereof, and the sheath being coupled to a central tube disposed within the tubular member such that pulling the central tube proximally pulls the sheath to release the expansion members and expand the tubular member;

FIG. 13 is a side cross-sectional view of another embodiment of a sheath configured to retain expansion members and a tubular member in an unexpanded configuration, the sheath being sealed at a distal end thereof such that a pull rod is used to pierce the distal end of the sheath after the tubular member has being inserted through tissue;

FIG. 14 is a side cross-sectional view of a trocar cannula having a tubular member extending therefrom and including an outer seal disposed around a portion of the tubular member for providing a seal therearound;

FIG. 15 is a side-cross-sectional view of a proximal end of the tubular member and outer seal of FIG. 14 showing the attachment of the tubular member and the outer seal to a housing of the cannula;

FIG. 16 is a perspective view of another embodiment of a trocar cannula having a tubular member extending therefrom, the tubular member including expansion members in the form of pleats extending longitudinally along the tubular member;

FIG. 17A is a cross-sectional view of the tubular member of FIG. 16 showing the pleats in an unexpanded configuration;

FIG. 17B is a cross-sectional view of the tubular member of FIG. 16 showing the pleats in an expanded configuration; and

FIG. 18 is a cross-sectional view of another embodiment of a tubular member including expansion members in the form of pleats, the pleats including locking features such that the pleats can be retained in the unexpanded configuration without the use of a sheath to retain the pleats.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

Various exemplary methods and devices are provided for dilating tissue using a trocar cannula having a flexible elongate tubular member. In general, the tubular member is configured to radially expand from an unexpanded configuration to an expanded configuration. As the tubular member moves to the expanded configuration, one or more expansion members that are incorporated into the tubular member are configured to maintain the tubular member in the expanded configuration. This allows the tubular member to dilate tissue when the tubular member is disposed through the tissue, thereby forming an enlarged passageway through the tissue, for example, for the insertion of instruments or other devices through the tubular member and into a body cavity.

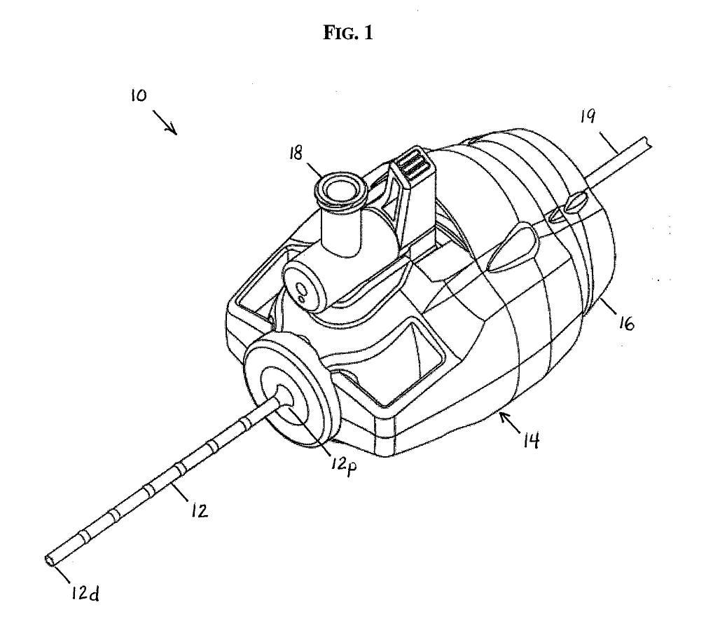

FIG. 1 illustrates one exemplary embodiment of a trocar cannula 10 for use with a trocar assembly. While not shown, the trocar cannula 10 can include an obturator that is slidably disposable therethrough, and that can have a distal end that extends beyond the distal end of the cannula 10 for penetrating through tissue. Once inserted through tissue, the obturator can be removed from the cannula 10 such that the cannula 10 provides a working channel through the tissue for inserting various instruments or other devices therethrough. As shown, the trocar cannula 10 generally includes a housing 14 having a flexible elongate tubular member 12 extending therefrom. The housing 14 can include a seal assembly 16 disposed therein that includes one or more seals that are configured to receive an instrument 19 or other devices therethrough. The housing 14 can also include an insufflation port 18 formed therein through which a body cavity can be insufflated. A person skilled in art will appreciate that, while the tubular member 12 is shown in connection with a trocar cannula 10, the tubular member 12 can be incorporated into virtually any device in which it is necessary to dilate tissue. For example, the tubular member 12 can be used with any device known in the art that is used to facilitate the creation of a working channel through tissue, and it need not include any housing coupled thereto.

The housing 14 can have any shape and size and it can be rigid or flexible depending on the intended use. The illustrated housing 14 includes an inner lumen formed therethrough that is aligned with an inner lumen of the tubular member 12 to allow instruments and other devices to be passed through the housing 14 and into the tubular member 12. As indicated above, the housing 14 can include a seal assembly having at least one seal disposed therein and effective to seal the working channel of the housing 14 when no instrument is disposed therein and/or to form a seal around an instrument disposed therethrough. The seal can be particularly useful during insufflation as it can prevent gases from escaping through the housing 14 and the tubular member 12. In particular, the seal can permit the passage of various other surgical instruments through the trocar cannula 10 while limiting or preventing the passage of fluid or gas therethrough. Various seals are known in the art, but typically the surgical access device can include at least one instrument seal that forms a seal around an instrument disposed therethrough, but otherwise does not form a seal when no instrument is disposed therethrough; at least one channel seal or zero-closure seal that seals the working channel when no instrument is disposed therethrough; or a combination instrument seal and channel seal that is effective to both form a seal around an instrument disposed therethrough and to form a seal in the working channel when no instrument is disposed therethrough. A person skilled in the art will appreciate that various seals known in the art can be used including, e.g., duckbill seals, cone seals, flapper valves, gel seals, diaphragm seals, lip seals, iris seals, etc. A person skilled in the art will appreciate, however, that the housing 14 is not necessary for use with the tubular member 12.

The tubular member 12 can have a variety of configurations, but in the illustrated embodiment the tubular member 12 is in the form of an elongate cylindrical tube defining an inner lumen extending therethrough and having a plurality of expansion members disposed along the length of the tubular member 12 such that the expansion members can radially expand the tubular member 12 from an unexpanded configuration to an expanded configuration. The tubular member 12 can be attached to the housing 14 using various techniques, such as by an adhesive, sealant, or any other attachment mechanism known in the art. For example, in one embodiment, the tubular member 12 can include a ring or other component, such as an O-ring, that can be coupled to a corresponding rib or other structure formed on the housing 14. An interlocking feature set, or an adhesive, or both, can be used to removably or permanently lock the tubular member 12 and the housing 14 together. The particular length of the tubular member 12 can vary, but the tubular member 12 preferably has a length in both the unexpanded and expanded configurations such that the tubular member 12 can extend through the tissue to form a working channel that provides access into a body cavity with a distal end 12d of the tubular member 12 extending into the body cavity. The diameter can also vary, but preferably the tubular member 12 has a diameter in the expanded configuration such that the inner lumen of the tubular member is configured to receive a variety of instruments and/or other devices therethrough. By way of non-limiting example, the tubular member 12 can have an unexpanded diameter in the range of about 12 mm to 15 mm, and a fully expanded diameter in the range of about 24 mm to 30 mm. The tubular member 12 can also have a diameter that is consistent along its length or portions of the tubular member can vary in diameter. For example, a proximal end 12p of the tubular member 12 can be flared outward to facilitate attachment to a housing, actuator, and/or other device.

The tubular member 12 can be formed from a variety of materials that allow the tubular member 12 to move between the unexpanded and expanded configurations to create a working channel through tissue. Preferably, the tubular member 12 is formed from a flexible and resilient mesh material, such as a woven or braided mesh, that allow the tubular member to move between the unexpanded and expanded configurations. The mesh material can be coated in an elastomer or other flexible material to provide a seal around the tubular member 12 while maintaining the flexibility of the tubular member 12. For example, the tubular member can be coated with isoprene, sanoprene, silicone, or polyurethane.

As explained above, the tubular member includes one or more expansion members incorporated therein that are configured to radially expand the tubular member into the expanded configuration and to maintain the tubular member in the expanded configuration with or without an instrument or other device disposed through the inner lumen of the tubular member. The expansion members can have a variety of configurations. In the embodiment shown in FIGS. 1-15, the tubular member 12 includes expansion members in the form of radially expandable rings 100 disposed within a wall of the tubular member 12. For example, the expandable ring 100 can be incorporated into a wall of the mesh material of the tubular member 12 during manufacturing of the tubular member 12 from the mesh such that the expandable ring 100 is internal or external to the mesh or woven therethrough, or the expandable ring 100 can be encapsulated during coating of the mesh material. Each expandable ring 100 can be configured to expand the tubular member 12 from an unexpanded configuration, for example having a first diameter D₁, to an expanded configuration, for example having a second diameter D₂, such that the tubular member 12 dilates tissue. Each ring 100 can, however, have a different diameter than the other rings 100. Any number of expandable rings 100 can be incorporated into the wall along the length of the tubular member 12. For example, in the illustrated embodiment shown in FIG. 1, the tubular member includes six expandable rings 100 disposed along the length of the tubular member 12. The rings can be disposed along the length of the tubular member 12 in any configuration. For example, the expandable rings 100 can be evenly spaced along the length of the tubular member 12, or the rings 100 can be positioned at different locations along the length of the tubular member.

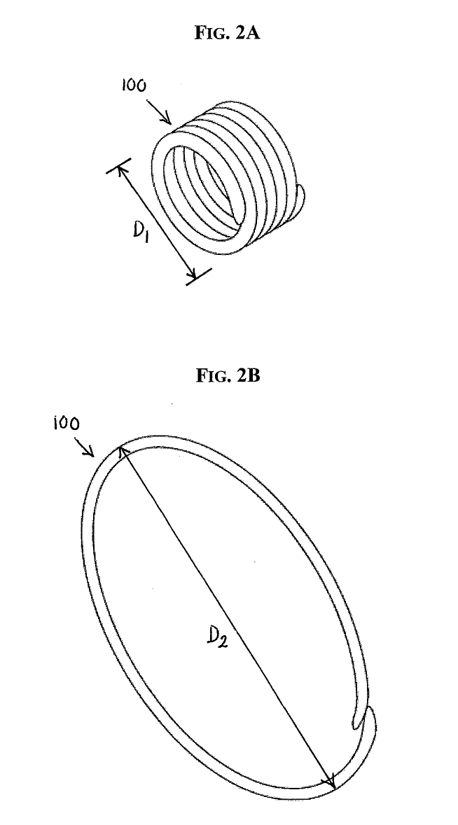

The radially expandable rings 100 can have a variety of configurations, but in one embodiment the expandable rings 100 are in the form of a coiled wire that is movable between an unexpanded and coiled configuration shown in FIG. 2A, and an expanded and uncoiled configuration shown in FIG. 2B. While the coiled rings are capable of being biased to either the unexpanded or expanded configuration, the coiled rings are prefereably biased to the expanded configuration such that the coiled rings can maintain the tubular member 12 in the expanded configuration. While the coiled wire forming the expandable ring 100 can be coiled any number of times to allow for different expanded diameters of the tubular member 12 in the expanded configuration, in the illustrated embodiment shown in FIG. 2A, the ring 100 is coiled six times such that the diameter D₂ of the tubular member in the expanded configuration is six times greater than the diameter D₁ of the tubular member in the unexpanded configuration. A person skilled in the art will appreciate however, that the ring 100 can be coiled any number of times, for example four times, such that the second diameter is approximately four times greater than the first diameter, depending on the desired size of the working channel through the inner lumen of the tubular member 12. In a preferred embodiment, D₁ can be in the range of 3 mm-5 mm, and D₂ can be in the range of 10 mm-15 mm. Moreover, a person skilled in the art will also appreciate that the expanded rings 100 can have any configuration that allows the rings to radially expand the tubular member.

The expandable rings 100 can be formed from a variety of materials that allow the expandable rings 100 to expand, for example, to uncoil, to move the tubular member 12 between the unexpanded and expanded configurations while also allowing the expandable ring 100 to maintain its expanded shape in the expanded configuration to maintain the radial expansion of the tubular member 12. Preferably, the expandable ring is formed from a material that is an expandable, elastic material. For example, the expandable ring 100 can be formed from a shape memory material, such as Nitinol, or from any other metal or plastic, spring stainless steel, polycarbonate, vectra, or composite polymer.

The tubular member 12 can include features to retain the expansion members in an unexpanded configuration during the insertion of the tubular member 12 through tissue and until such time as it is desired to radially expand the expansion members and tubular member 12 to create the working channel through tissue. While the tubular member 12 can include various features to retain the expansion members in the unexpanded configuration, in one embodiment the tubular member can include a sheath. The sheath can have a variety of configurations and can be disposed at various locations.

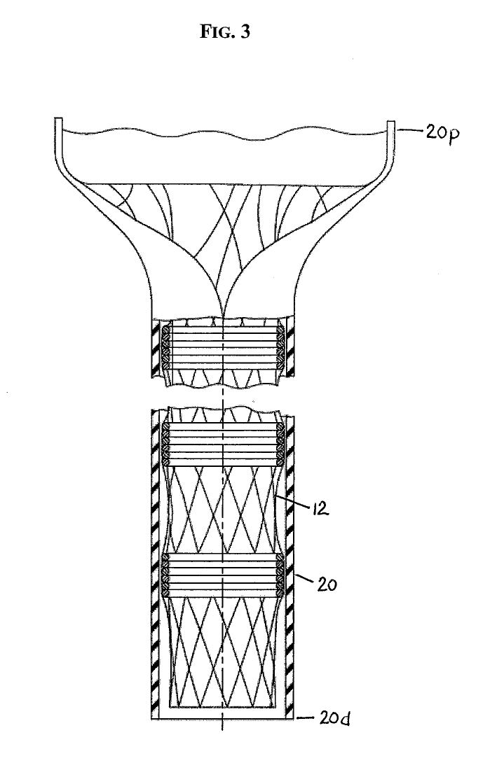

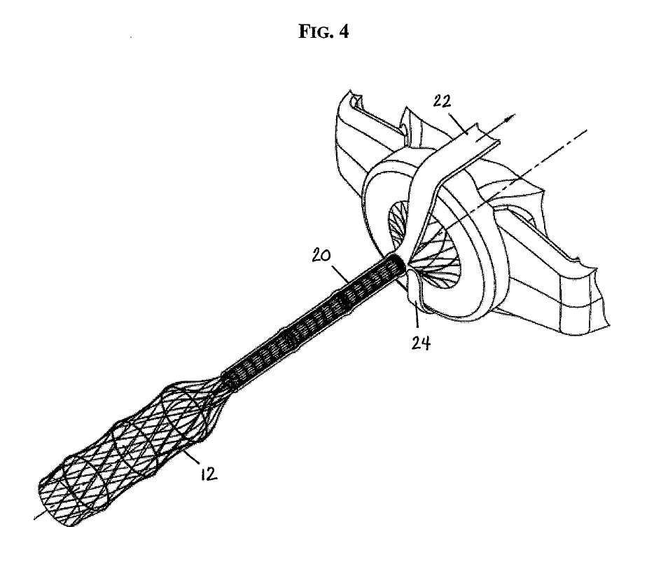

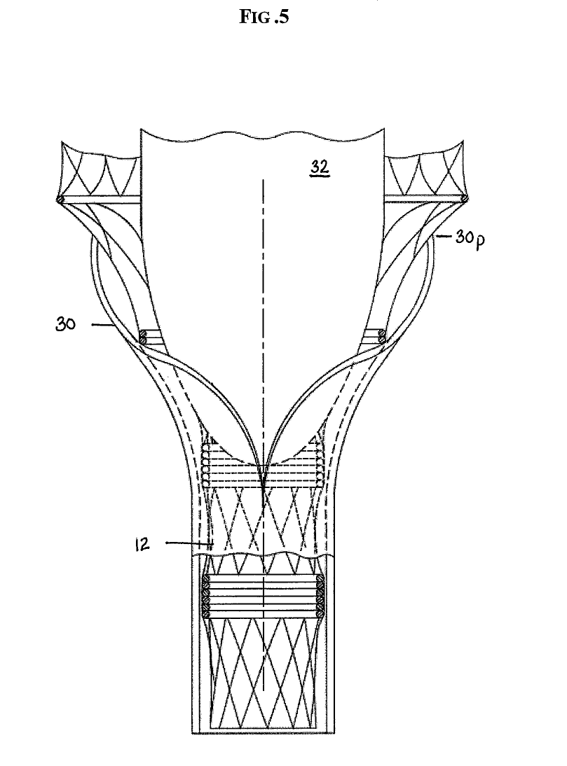

In one embodiment illustrated in FIGS. 3-4, the tubular member 12 includes a sheath 20 in the form of an elongate cylindrical tube that is disposed around an outer surface of the tubular member 12 and that is configured to retain the expansion members in an unexpanded configuration such that removal of the sheath 20 allows the expansion members to radially expand, thus radially expanding the tubular member 12. The sheath 20 is not attached to the tubular member 12 to allow removal of the sheath 20 from the tubular member 12, and the sheath 20 is slidable along the length of the tubular member 12 to allow the sheath 20 to be removed. The sheath 20 can be held in place along the outer surface of the tubular member 20 in a variety of ways, including, for example, with an interference fit around the tubular member 12. The length of the sheath 20 can vary, but it preferably has a length that corresponds to a length of the tubular member 12, or that at least extends around all of the expansion members disposed within the wall of the tubular member 12. Thus, the sheath 20 can extend along the entire length of the tubular member 12, or the sheath 20 can extend along a portion of the length. The diameter of the sheath 20 can vary, but it is preferably configured to allow the sheath 20 to fit around the outer diameter of the tubular member 12 when the tubular member 12 is in the unexpanded configuration. The diameter can be constant along its entire length or it can vary depending on the shape and diameter of the tubular member 12. The sheath 20 can be formed from a variety of materials, but preferably it has at least enough strength to retain the expansion members in the unexpanded configuration. For example, the sheath can be formed from plastic, silicone, polyurethane, isoprene, or sanoprene. In certain embodiments, the sheath 20 can be formed from a material that is puncturable or that tears easily.

In order to facilitate removal of the sheath 20 from the tubular member 12, in one embodiment shown in FIG. 4, the sheath 20 can be split at the point of attachment of the tubular member 12 to the housing 14 such that one or more tabs can extend along the outside of the housing 14 of the trocar cannula 10. The tabs can be used to pull the sheath 20 proximally to remove it from the outer surface of the tubular member 12. For example, as shown in FIG. 4, the sheath 20 can be split into two halves such that the sheath 20 includes first and second tabs 22, 24. As the tabs 22, 24 are pulled proximally, the sheath 20 continues to separate. In particular, the sheath 20 can simply tear as the tabs 22, 24 are pulled proximally, or it can separate along a predetermined location, such as along perforations extending longitudinally along the sheath 20. As the sheath 20 moves proximally along the length of the tubular member 12, the distal-most expansion member is exposed and allowed to move to the expanded configuration, as shown in FIG. 4. Each successive expansion member is released from the retention of the sheath 20 as the sheath 20 moves proximally until the sheath 20 is removed from the outer surface of the tubular member 12 and all of the expansion members are exposed and in the expanded configuration. Thus, the entire length of the tubular member 12 is radially expanded to dilate tissue and form a working channel therethrough.

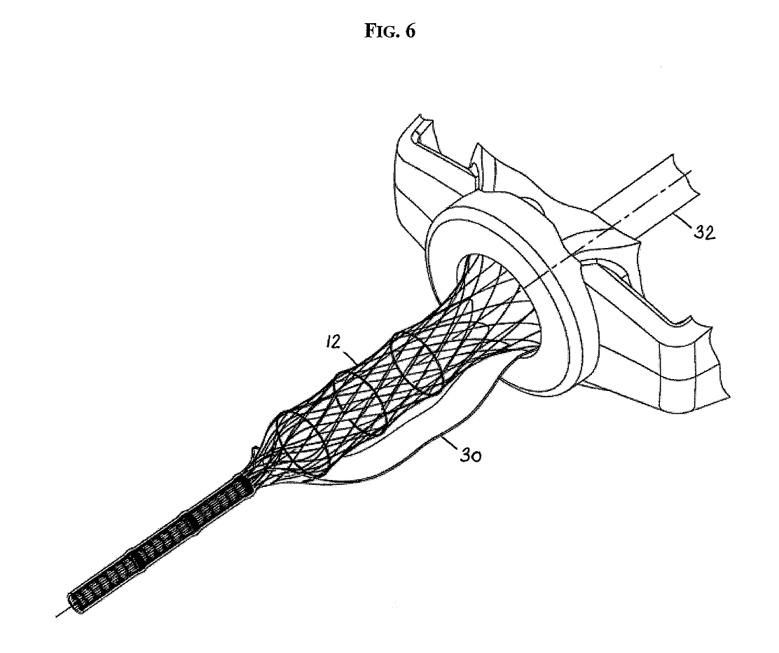

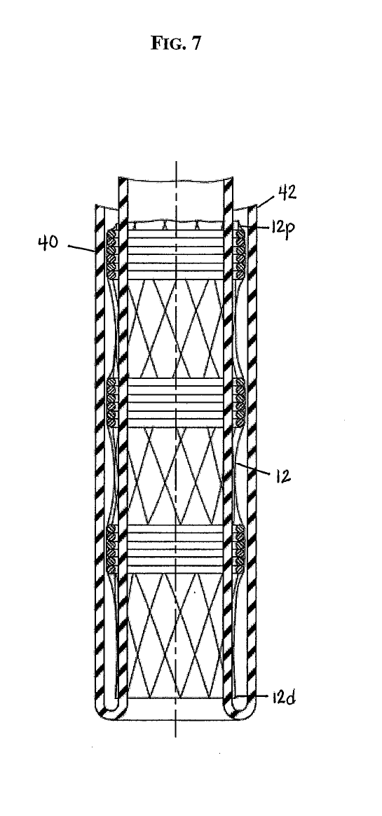

Rather than slidably removing the sheath from around the outer surface of the tubular member 12, a sheath disposed around the tubular member can function in a variety of other ways to retain and then release the expansion members to allow radial expansion of the tubular member 12. In another embodiment illustrated in FIGS. 5-6, a sheath 30 is disposed around an outer wall of the tubular member 12 as described above, and the sheath 30 is configured to rupture as a tool 32 or other device is inserted through the inner lumen of the tubular member 12 such that the rupture of the sheath 30 releases the expansion members to allow the tubular member 12 to radially expand and dilate tissue. In particular, as the tool 32 moves distally through the tubular member 12, the sheath 30 begins to rupture at it proximal end 30p first such that the proximal-most expansion member can move into the expanded configuration, as shown in FIG. 6. Each successive expansion member is released from the retention of the sheath 30 as the tool 32 moves distally though the tubular member 12 and the sheath 30 continues to rupture along its length until substantially the entire length of the sheath 30 is ruptured such that all of the expansion members are in the expanded configuration and the entire length of the tubular member 12 is radially expanded to dilate tissue and form a working channel therethrough. While not shown, the sheath 30 can optionally include a pre-defined rupture location. For example, the sheath 30 can be perforated along its length such that the sheath 30 will tear along the perforation as the tool 32 is moved through the inner lumen of the tubular member 12.

In yet another embodiment, the sheath and/or various other optional components can be slidably disposed around portions of the outer and inner walls of the tubular member such that the expansion members are retained in the unexpanded configuration and additional structural support is provided to the tubular member to assist in retaining the shape of the tubular member, for example, to prevent inversion of a distal end of the tubular member during tool removal.

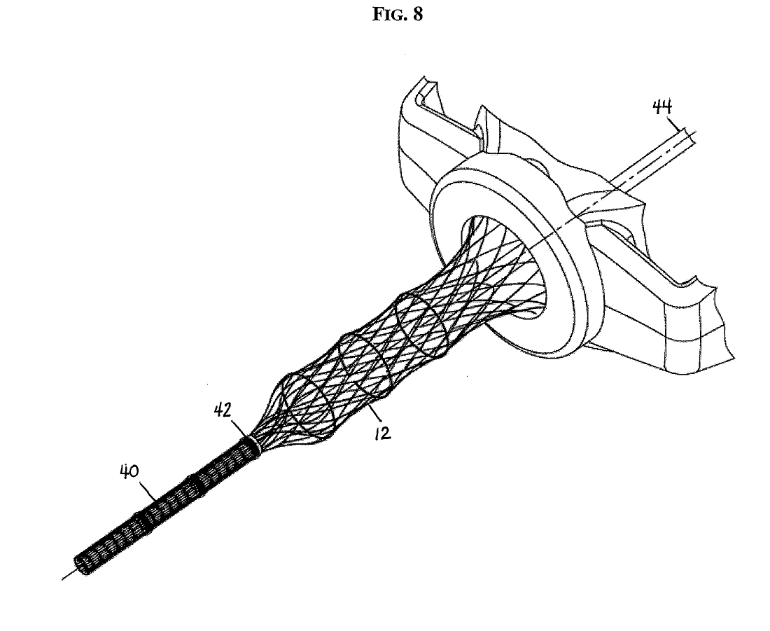

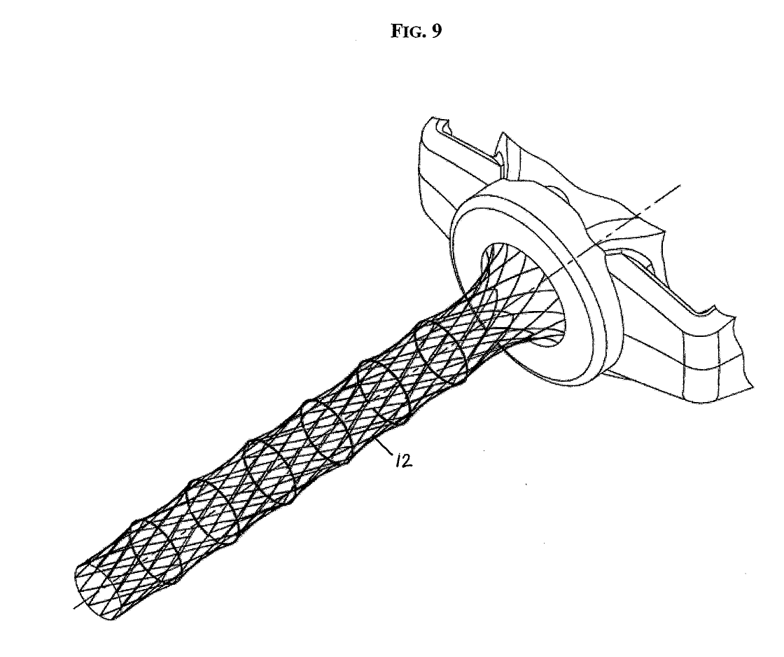

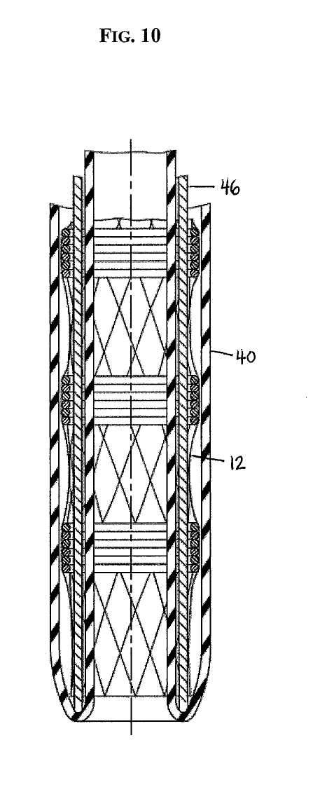

In the embodiment illustrated in FIGS. 7-9, the sheath 40 is in the form of an elongate cylindrical tube that is disposed continuously around an outer surface of the tubular member 12, around the distal end 12d of the tubular member 12, and through the inner lumen of the tubular member 12 such that a first end 42 of the sheath 40 is positioned around the outer surface of the proximal end 12p of the tubular member 12 and a second end 44 of the sheath 40 is disposed within the proximal end 12p of the tubular member 12 and extends proximally from the housing of the trocar cannula. The sheath 40 is configured to retain the expansion members such that removal of the sheath 40 allows the expansion members to radially expand, thus radially expanding the tubular member 12. In order to remove the sheath 40, the second end 44 of the sheath 40 that extends through and out the proximal end of the housing can be grasped by a user. As the second end 44 of the sheath 40 is pulled in a proximal direction, the inner portion of the sheath 40 moves proximally and the outer portion of the sheath 40 moves distally such that the proximal-most expansion member moves into the expanded configuration first, as shown in FIG. 8. Each successive expansion member is released by the outer portion of the sheath 40 as the second end 44 of the sheath is pulled proximally until all the expansion members are released and moved into the expanded configuration. Continuing to pull the second end 44 of the sheath 40 proximally causes the outer portion of the sheath 40 to be pulled inside the inner lumen of the tubular member 12 until the entire sheath 40 is removed from the inside of the tubular member 12, as shown in FIG. 9.

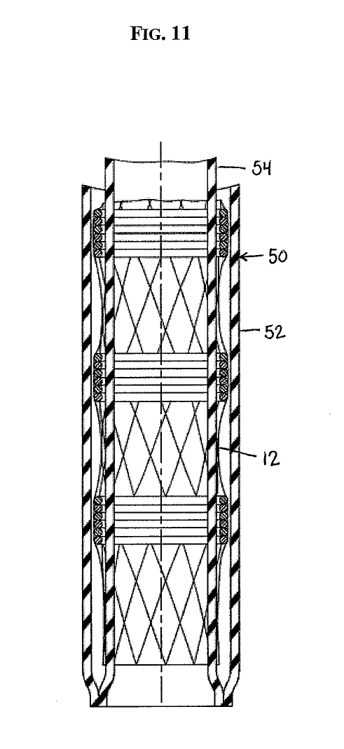

The tubular member can also be used in conjunction with other components to facilitate retention of the expansion members and/or provided added rigidity to the tubular member. For example, the tubular member can optionally include a central tube 46, as shown in FIG. 10, that can be disposed within the inner lumen of the tubular member 12 between the inner surface of the tubular member 12 and the inner portion of the sheath 40. The central tube 46 can extend along the entire length of the tubular member 12 or along any portion thereof. The central tube 46 can be advantageous as it can provide added rigidity to the tubular member and can assist in preventing inversion of the tubular member as the sheath is removed, for example, if the tubular member is not stiff enough without the central tube or if the distal end of the tubular member is too sharp to allow removal of the sheath without puncturing the sheath. Thus, the sheath 40 can extend around a distal end of the central tube 46 instead of around a distal end of the tubular member 12 so that the sheath 40 can be pulled proximally without inverting the tubular member 12. A person skilled in the art will appreciate that, while the central tube 40 is shown in conjunction with the sheath 40, the central tube can be used with any embodiment disclosed herein.

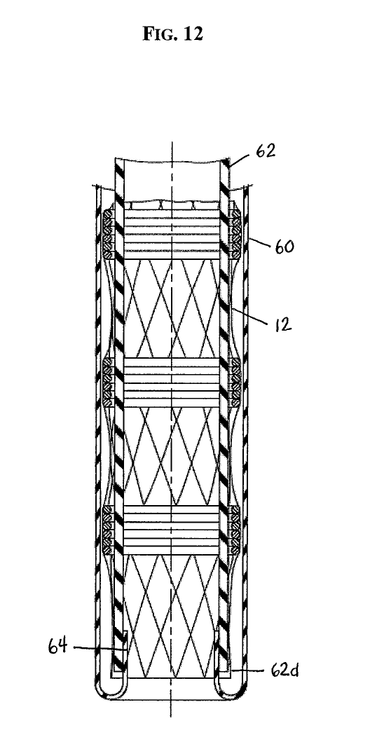

In another embodiment, the sheath can be formed from more than one component that can be joined together during manufacturing. For example, as illustrated in FIG. 11, a sheath 50 can be formed from an outer portion 52 that extends along an outer surface of the tubular member 12 and an inner portion 54 that extends through the inner lumen of the tubular member 12. The distal ends of the outer portion 52 of the sheath 50 and the inner portion 54 of the sheath 50 are joined, as shown in FIG. 11, such that as a proximal end of the inner portion 54 of the sheath 50 is pulled in a proximal direction, the inner portion 54 of the sheath 50 moves proximally and the outer portion 52 of the sheath 50 moves distally to allow the proximal-most expansion member to move into the expanded configuration first as the outer portion 52 of the sheath 50 moves distally and releases the expansion members. The distal ends of the outer and inner portions 52, 54 of the sheath 50 can be joined together using a variety of techniques, such as thermal sealing.

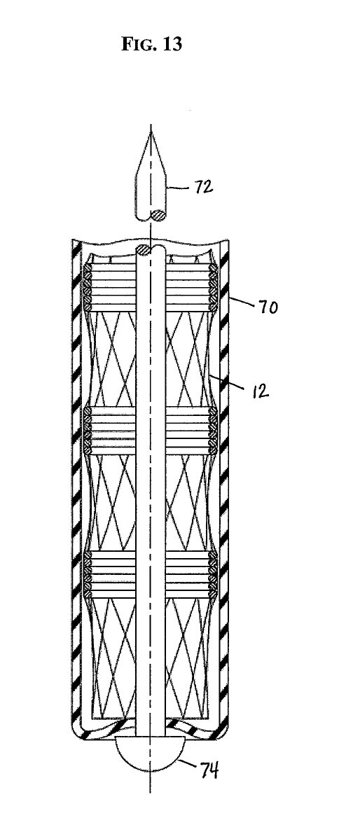

In another embodiment illustrated in FIG. 12, a sheath 60 extends along the outer surface of the tubular member 12 and around a distal end thereof and into a distal portion of the inner lumen of the tubular member 12. The tubular member 12 includes a central tube 62 disposed therethrough that is configured to removably couple at a distal end 62d thereof to an end 64 of the sheath 60 that extends into the distal portion of the tubular member 12. The end 64 of the sheath 60 extending into the inner lumen of the tubular member 12 can be mated to the distal end 62d of the central tube 62 such that the central tube 62 can be pulled proximally out of the tubular member 12 to pull the sheath 60 into the lumen of the tubular member 12. The trailing end of the sheath 60 will thus move along the outer surface of the tubular member 12 in a distal direction such that the proximal-most expansion member is released to move into the expanded configuration. Each successive expansion member is released by the outer portion of the sheath 60 as the central tube 62 is pulled proximally until all the expansion members are released and moved in the expanded configuration. The sheath 60 and the central tube 62 can be coupled to one another in a variety of ways using any method known in the art. For example, there can be a thermal or chemical bond that attaches the sheath 60 to the central tube 62.

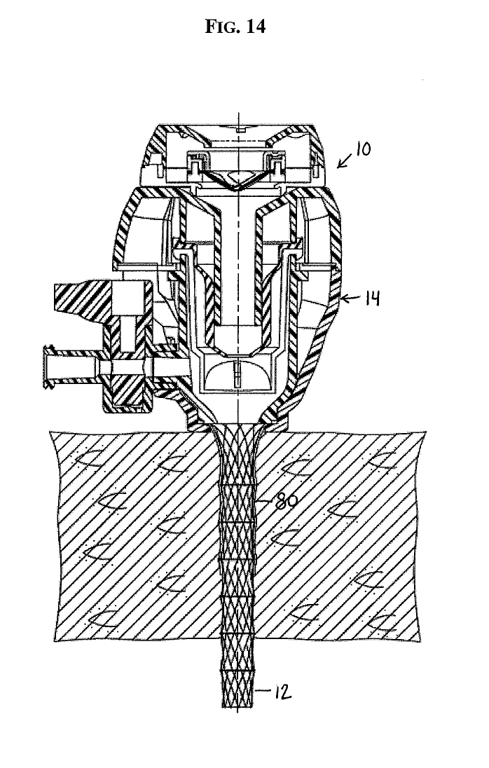

In yet another embodiment shown in FIG. 13, a sheath 70 can be formed around the outer surface of the tubular member 12 and can be sealed around the distal end thereof to fully seal the working channel. This allows the sheath 70 to aide in maintaining a seal through the tubular member 12 as the tubular member 12 is inserted through tissue and into a body cavity. In one embodiment, in order to pull the sheath 70 proximally to release the expansion members in the tubular member 12, a pull rod 72 can be provided. For example, as shown in FIG. 13, a pull rod 72 can be disposed through the tubular member 12 during the manufacturing process such that the rod 72 extends through the tubular member 12, while a head 74 of the rod 72 is positioned on the outside of the distal end of the sheath 70 such that pulling proximally on a proximal end of the pull rod 72 will cause the head 74 of the pull rod 72 to pull the sheath 70 proximally to remove the sheath 70 from the outer surface of the tubular member 12. The diameter of the pull rod 72 can also vary, but preferably the pull rod has a diameter such that the pull rod 72 can be positioned within the inner lumen of the tubular member 12 when the tubular member 12 is in the unexpanded configuration. In another embodiment, a pull rod can have a pointed tip formed on the distal end of the head such that the pull rod can pierce through tissue for inserting the trocar cannula therethrough. Thus, the pull rod can function as an obturator and a removal device for removing the sheath 70 from the outer surface of the tubular member 12.

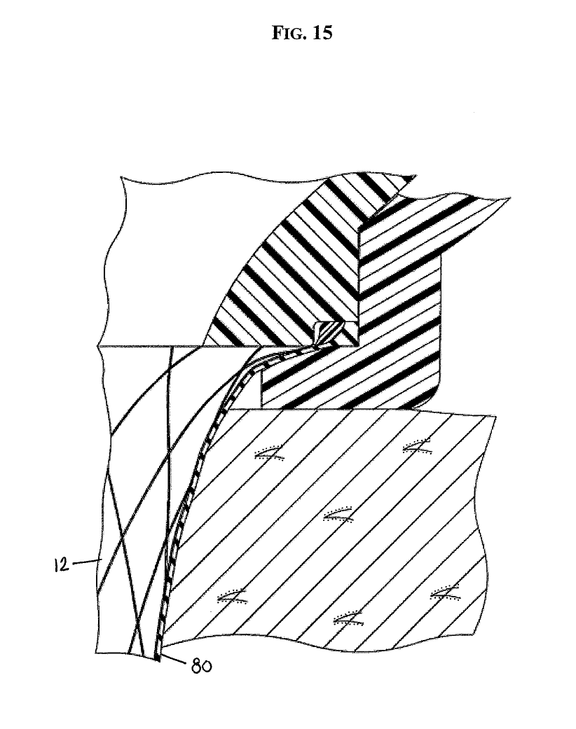

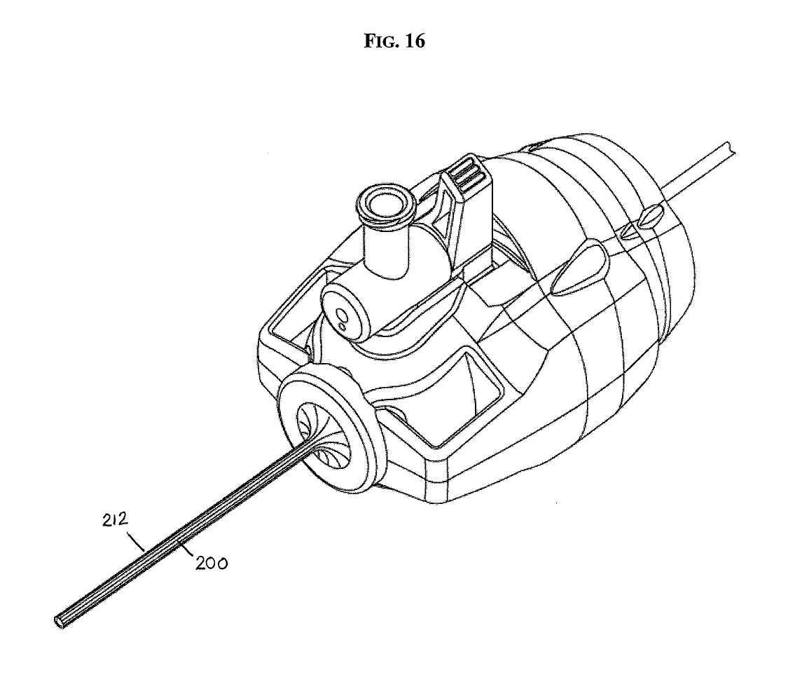

The tubular member can also optionally include other features adapted to provide a seal between the tubular member and the tissue through which the tubular member is disposed to create the working channel. For example, the tubular member 12 can include an outer seal 80 in the form of a flexible, expandable sheath disposed around a least a portion of the outer wall of the tubular member 12, as shown in FIGS. 14-15. The outer seal 80 can have a variety of configurations, and can vary in length such that the outer seal 80 can extend along an entire length of the tubular member 12, or the outer seal can extend from the proximal end of the tubular member 12 to any location proximal of the distal end of the tubular member 12. As shown in FIG. 14, the outer seal 80 extends from the proximal end of the tubular member 12 to a location substantially in the midpoint of the tubular member 12. The outer seal 80 can be formed from a variety of materials, but preferably is formed from a material that provides a fluid seal around the tubular member 12 but that is flexible such that the outer seal 80 can radially expand as the tubular member 12 radially expands when the expansion members disposed therein move into the expanded configuration. The outer seal 80 can be coupled to the trocar cannula at various locations, but in the embodiment shown in FIG. 15, the outer seal 80 is coupled to the tubular member 12 at the same attachment point as the proximal end of the tubular member 12 such that the seal 80 extends all the way up to the proximal end of the tubular member 12. In another embodiment, the outer seal 80 can be formed as a separate sleeve from the tubular member 12, or the outer seal 80 can be integrally formed within the mesh of the tubular member 12.

While the expandable rings 100 are described above as being retained in the unexpanded configuration by a sheath, such as the sheaths described above, various other techniques can be used to move the expandable rings 100 between the unexpanded configuration and the expanded configuration. For example, an axial coil member can be incorporated into the tubular member such that coil member is associated with each of the expandable rings 100 incorporated into the wall of the tubular member. The axial coil member can control the movement of the expandable rings 100 between the unexpanded and expanded configuration using, for example, a twisting mechanism or a cam mechanism to uncoil the expandable rings 100 and radially expand the tubular member. The use of this type of mechanism to control the expandable rings 100 allows the rate of the expansion of the expandable ring 100 to be controlled, as well as the diameter to which the expandable rings 100 are expanded. Thus, the diameter of the tubular member and the rate of dilation of the tissue can be varied and precisely controlled. In another embodiment, the axial coil member alone can function as the expansion member to radially expand the tubular member.

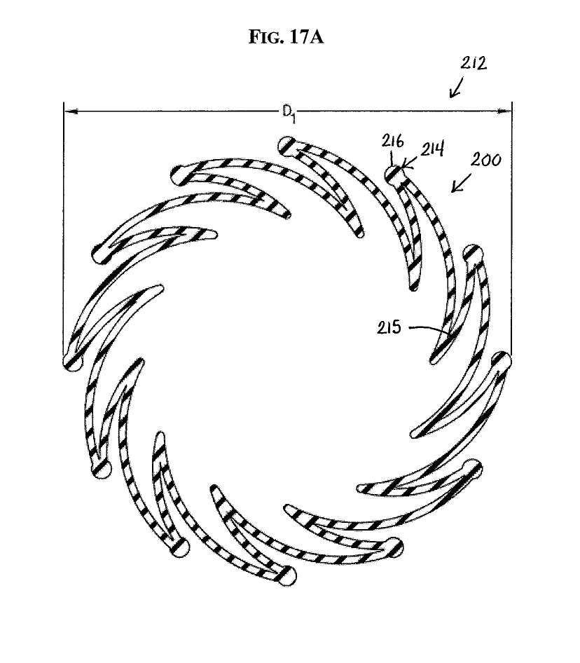

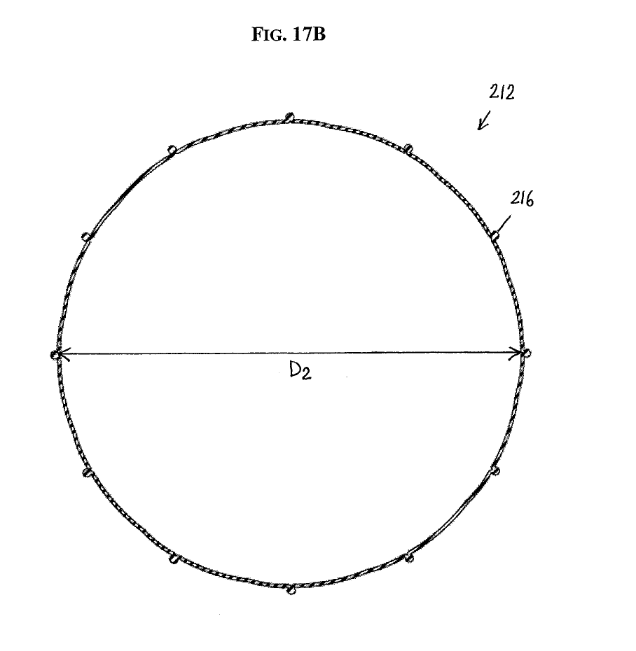

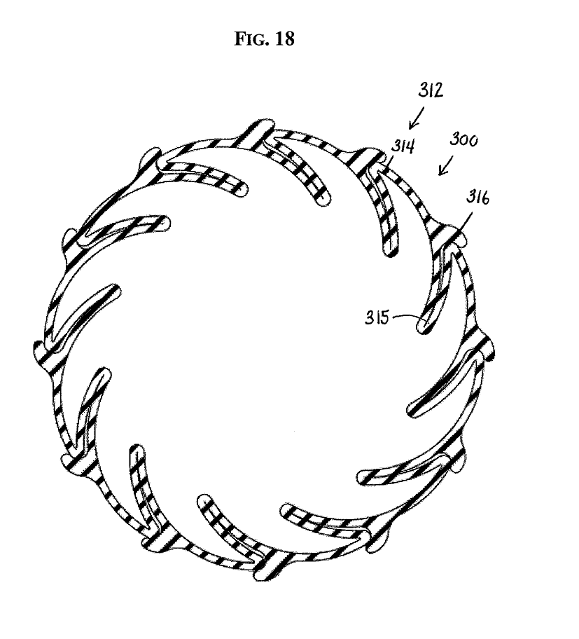

Various other configurations of expansion members can be used to radially expand the tubular member. In another embodiment illustrated in FIGS. 16-18, the expansion members can be in the form of a plurality of pleats 200 formed in the wall of a tubular member 212 and extending longitudinally along the tubular member 212. Each pleat 200 can be formed by folding portions of the wall of the tubular member back upon itself to create longitudinally-extending peaks 214 and troughs 215. As shown in FIG. 17A, each peak 214 can optionally include a rib 216 formed thereon, with the rib 216 being configured to provide longitudinal stiffness to the tubular member 212. The shape and size of the ribs 216 can vary depending on the desired stiffness of the tubular member 212, but in an exemplary embodiment they have a cylindrical shape with a diameter that is at least twice the thickness of the wall of the tubular member 212. In use, the pleats 200 are configured to allow the tubular member 212 to move from the unexpanded configuration, for example having a first diameter D₁, to the expanded configuration, for example having a second diameter D₂, such that the tubular member 212 dilates tissue. Any number of pleats 200 can be incorporated into the wall along the length of the tubular member 212, and each pleat 214 can have any height h, depending on the desired diameter of the tubular member 212 in the expanded configuration.

In one embodiment, the pleats can be biased to the expanded configuration. Thus, any of the sheaths described above can be used to retain the pleats in the unexpanded configuration. In another embodiment, the pleats can include additional features to retain the pleats in the unexpanded configuration without the need for a sheath. In one embodiment illustrated in FIG. 18, the pleats 300 can interlock with one another to maintain the tubular member in the unexpanded configuration. Insertion of an instrument or other device through the inner lumen of the tubular member 312 can cause the pleats 300 to unlock from one another and allow the tubular member 312 to radially expand. The pleats 300 can interlock in a variety of ways, but in the illustrated embodiment each pleat 300 includes a longitudinally-extending ridge 316 formed at a midpoint between the peak 314 and the trough 315. Each ridge 316 is configured to overlap an adjacent peak 314. The ridges 316 can have a variety of configurations, but in the illustrated embodiment the ridges 316 are in the form of C-shaped or hook-shaped members extending from the pleats 300 and having a shape and size for removably associating with the adjacent peaks. In this way, the peaks 314 of each pleat 300 are interlocked with the ridges 316 of the adjacent pleats 300 such that the pleats 300 are retained in the unexpanded configuration without the need for any additional retention features, such as a sheath. A person skilled in the art will appreciate that the pleats 300 can have various configurations that allow the pleats 300 to retain themselves in the unexpanded configuration. Moreover, a person skilled in the art will appreciate that a variety of other types of expansion members can be used that are configured to retain themselves in the unexpanded configuration.

A method for dilating tissue is also provided herein. A person skilled in the art will appreciate that the tubular member described herein can be used in any procedure, and that the trocar cannula is merely discussed as an example of a device that can utilize the tubular member. In one embodiment, a trocar cannula can be inserted through tissue, for example, using the tip of an obturator. In particular, a small incision can be made in the skin, and an obturator can be inserted through the cannula and the entire assembly can be inserted through tissue with the tubular member in the unexpanded configuration. Other techniques that do not utilize the obturator can also be used, including inserting the trocar cannula directly through the incision. The proximal end of the tubular member can be positioned proximal of or with the tissue wall through which the tubular member has been inserted, and the distal end of the tubular member can be positioned within a body cavity, such as in the peritoneal cavity. The tubular member can then be radially expanded to dilate the tissue therearound. In particular, any of the sheaths described above can be removed from the tubular member, for example, by pulling the sheath or a tool attached to the sheath proximally. In other embodiments, the sheath can be ruptured by inserting an instrument or other device through the tubular member. This causes the expansion members disposed in the tubular member to radially expand the tubular member, thus dilating the tissue disposed around the tubular member. In other embodiments, the expansion members can radially expand the tubular member as an instrument or other device is inserted through the tubular member. For example, the expansion members can be in the form of pleats that are configured to interlock such that the pleats remain in the unexpanded configuration until an instrument is inserted through the tubular member to expand the pleats. The expansion members can provide rigidity to the tubular member to prevent the tubular member from collapsing when no instrument is disposed therein.

The devices disclosed herein can also be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the invention described herein will be processed before surgery. First, a new or used instrument is obtained and if necessary cleaned. The instrument can then be sterilized. In one sterilization technique, the instrument is placed in a closed and sealed container, such as a plastic or TYVEK bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that device is sterilized. This can be done by any number of ways known to those skilled in the art including beta or gamma radiation, ethylene oxide, steam, and a liquid bath (e.g., cold soak).

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. A trocar cannula, comprising:
an elongate tubular member defining an inner lumen extending therethrough and configured to receive a surgical instrument therethrough, the tubular member being configured to radially expand to increase an inner diameter of the inner lumen when an instrument is disposed within the inner lumen, and the tubular member having an expansion element incorporated therein and configured to maintain the tubular member in a radially expanded position when no instrument is disposed within the inner lumen.

2. The trocar cannula of claim 1, wherein the expansion element is biased to an expanded position.

3. The trocar cannula of claim 2, wherein the tubular member includes a sheath disposed therearound and configured to retain the expansion element in an unexpanded configuration such that removal of the sheath allows the expansion element to radially expand.

4. The trocar cannula of claim 3, wherein the sheath includes a closed distal end to provide a seal for the tubular member as the tubular member is inserted through tissue.

5. The trocar cannula of claim 1, wherein the tubular member is formed from a mesh material.

6. The trocar cannula of claim 5, wherein the expansion element comprises at least one radially expandable ring disposed within a wall of the mesh material.

7. The trocar cannula of claim 6, wherein the at least one radially expandable ring comprises a coiled wire.

8. The trocar cannula of claim 6, wherein the at least one radially expandable ring is configured to expand from a first diameter to a second diameter that is four times greater than the first diameter.

9. The trocar cannula of claim 1, wherein the tubular member includes an internal sheath disposed therein and configured to provide resiliency to a distal end of the tubular member to prevent inversion of the distal end of the tubular member when the tubular member is in the radially expanded position.

10. The trocar cannula of claim 9, wherein the internal sheath extends from inside the tubular member and around the distal end of the tubular member.

11. The trocar cannula of claim 1, wherein the expansion element comprises a plurality of pleats formed in the tubular member and extending longitudinally along the tubular member.

12. The trocar cannula of claim 11, wherein each pleat includes a peak having a rib formed thereon and extending longitudinally along the tubular member, the ribs being configured to provide longitudinal stiffness to the tubular member.

13. The trocar cannula of claim 11, wherein the pleats are configured to interlock with one another to maintain the tubular member in an unexpanded position, and wherein insertion of an instrument through the inner lumen of the tubular member is configured to cause the pleats to unlock to allow radial expansion of the tubular member.

14. The trocar cannula of claim 1, wherein a proximal end of the tubular member is mated to a housing having an inner lumen extending therethrough and aligned with the inner lumen of the tubular member, and at least one seal disposed therein and configured to form a seal around an instrument inserted therethrough.

15. A method of dilating tissue, comprising:
inserting a radially expandable cannula into tissue such that an inner lumen of the cannula defines an opening extending through the tissue and into a body cavity;
inserting an instrument through the inner lumen of the cannula, the instrument causing the radially expandable cannula to expand into an expanded configuration wherein a diameter of the inner lumen is increased; and
removing the instrument from the inner lumen of the cannula, wherein an expansion element integrally formed with the radially expandable cannula maintains the cannula in the radially expanded configuration.

16. The method of claim 15, wherein the expansion member is a radially expandable ring disposed within a wall of the cannula, the ring being biased to the expanded configuration, and wherein the radially expandable ring expands from a first diameter to a second greater diameter upon insertion of the instrument through the inner lumen of the cannula.

17. The method of claim 15, wherein the expansion member is a plurality of pleats formed in the cannula that extend longitudinally along the cannula, and wherein each pleat includes a peak having a rib formed thereon that provides longitudinal stiffness to the tubular member upon insertion of the instrument through the inner lumen of the cannula.

18. A method of dilating tissue, comprising:
inserting a radially expandable cannula into tissue such that an inner lumen of the cannula defines an opening extending through the tissue and into a body cavity, the cannula including at least one expansion element biased to an expanded position; and
removing a sheath disposed around an outer surface of the cannula such that the expansion element expands to cause the radially expandable cannula to expand into an expanded configuration wherein a diameter of the inner lumen is increased.
